(19) United States
(12) Patent Application Publication
(10) Pub. No.: US 2011/0245888 A1
(43) Pub. Date: Oct. 6, 2011

(54) MEDICAL DEVICE WITH CHARGE LEAKAGE DETECTION

(75) Inventors: Steven W. Badelt, Granada Hills, CA (US); Gabriel A. Mouchawar, Valencia, CA (US); George I. Isaac, Port Hueneme, CA (US); Neal Forss, East Palo Alto, CA (US); Lyle Frank Weaver, Woodside, CA (US)

(73) Assignee: PACESETTER, INC., Sylmar, CA (US)

(21) Appl. No.: 12/754,491

(22) Filed: Apr. 5, 2010

(51) Int. Cl. A61N 1/39

(52) U.S. Cl. 607/6

(57) ABSTRACT

A medical device (implantable or external) is provided that comprises a power source, a charge storage member, a terminal connector, a switch network, a controller and a leak detection module. The charge storage member is configured to receive and store energy from the power source. The terminal connector is configured to be coupled to a lead to be implanted in a patient proximate to tissue of interest. The switch network is electrically disposed between the charge storage member and the terminal connector. The switch network changes between open and closed states to disconnect and connect the charge storage member and the terminal connector. The controller controls storage of energy in the charge storage member and delivery of stimulating pulses from the charge storage member to the lead coupled to the terminal connector. The leak detection module obtains a leakage measurement by sensing at least one of i) a voltage potential of the charge storage member and ii) current flow from the charge storage member. The leak detection module compares the leakage measurement to a leakage threshold to determine when the leakage measurement satisfies the leakage threshold.

### FIELD OF THE INVENTION

Embodiments of the present invention relate generally to medical devices that utilized charge storage members for treating various cardiac, physiologic and neurologic disorders. More particularly, embodiments of the present invention relate to implantable or external medical devices with leakage detection circuitry to detect leakage of energy to tissue and with leakage prevention circuitry to take corrective action based upon detection of energy leakage.

### BACKGROUND OF THE INVENTION

Numerous medical devices exist today, including but not limited to electrocardiographs (“ECGs”), electroencephalographs (“EEGs”), squid magnetometers, implantable pacemakers, implantable cardioverter-defibrillators (“ICDs”), neurostimulators, electrophysiology (“EP”) mapping and radio frequency (“RF”) ablation systems, and the like (hereafter generally “implantable medical devices” or “IMDs”. IMDs commonly employ one or more conductive leads that either receive or deliver voltage, current or other electromagnetic pulses (generally “energy”) from or to an organ or tissue (collectively hereafter “tissue”) for diagnostic or therapeutic purposes.

Certain types of IMDs include internal charge storage members, such as one or more capacitors. The charge storage members are connected to a switch network also referred to as a bridge. The bridge includes a network of transistors that are controlled by a processor to open and close in different combinations to deliver stored energy from the charge storage members to the tissue through the electrodes.

However, the potential exists that electrical components along the conductive path between the charge storage members and the electrodes may experience electrical failure. For example, the bridge may become damaged when a high voltage shock is delivered over a lead that has a faulting electrode or conductor(s) therein. When a lead undergoes a fault, the conductive wires within the lead may be directly shorted to one another or may become directly shorted to the housing of the IMD 10 (which also may serve as a shocking electrode). When one or more electrodes are in a short circuit condition, the current of a high voltage shock from the charge storage members does not discharge into the normally expected resistive load of tissue, such as the heart. Without the resistive load of the tissue to absorb the current, a large voltage potential builds up across the bridge. In this instance, the high voltage potential from the capacitors is applied directly across the bridge. While the transistors in the bridge are well suited to carry high current, these transistors are not designed to withstand large voltage potentials at high current. When a large voltage potential is created across one or more of these transistors, this may damage one or more of the transistors in the bridge.

Alternatively, a lead may be operating normally, but receive a large voltage from an external source such as from an external defibrillator. External defibrillation may induce over 1000 V on a lead. This high voltage potential may also damage the transistors in the bridge. When the transistors in the bridge damage, the potential exists that the bridge can no longer isolate the charge storage members from the tissue. Without electrical isolation, as soon as the charge storage members begin to charge, current leaks from the charge storage members to the tissue of interest. Current leakage from high voltage energy storage capacitors of an IMD 10 may occur due to the reasons noted above as well as due to various other reasons such as, for example, defective components in the IMDs, components damaged during the handling process, electrical overstress by the erroneous implantation of the IMDs into the patient's heart by the surgeons, semiconductor contamination of the switching devices of the IMD.

When the bridge experiences a failure, one or more of the switching transistors may permanently enter an open circuit state or a closed circuit state. When certain combinations of the switching transistors fail in a closed circuit state, the potential exist that the charge storage members become directly and permanently connected to the connector terminals that are joined to one or more electrodes. Thus, as soon as the IMD 10 begins to charge the charge storage members, spurious current may flow from the charge storage members through the lead and be delivered to the tissue surrounding the electrodes. It is mandated by International Standards that medical devices do not inject spurious current into the patient beyond certain limits. First, spurious current flow may promote electrode corrosion or electroplating on the electrodes. Second, spurious current may stimulate the surrounding tissue at a time when stimulation is not needed or desired.

IMDs may charge the energy storage capacitors periodically even when a patient does not need therapy. For example, various capacitors, such as the commonly employed aluminum electrolytic capacitors, are typically charged to full voltage every couple of months to prevent performance degradation. Whether energy is actually required to perform capacitor reformation depends upon whether the patient receives relatively frequent defibrillation shocks. IMDs that do not periodically receive at least one defibrillation shock may receive a periodic cycle of capacitor reformation. IMDs that receive at least a defibrillation shock every month or two; however, do not typically require such periodic capacitor reformation because such capacitor reformation is achieved automatically during the generation of the defibrillation shocks.

In general, the amount of allowable leakage currents depends on various design configurations of the implanted electrodes of the IMDs such as implant positions of such electrodes, surface areas thereof, and so forth. A current density for cardiac stimulation by the direct contact electrodes has been determined to be about 1.5 mA/cm², below which it is very unlikely for excitable cardiac tissues to be stimulated. IMDs generally include large-area electrodes for high voltage shock therapies as well as small-area electrodes for pacing and sensing, where each has its own limit for the allowable current leakage.

In accordance with certain standards, the allowable current leakage from the high-voltage (HV) cardiac electrodes under normal operating conditions is limited to 1 uA when the HV capacitors are discharged and 10 uA when such capacitors are charged. When the output switches of the IMDs leak electric current (e.g., through various electrodes thereof) in an amount less than the foregoing standards, cardiac tissues around the leaking electrodes do not generally respond to such direct current and, thus, do not exhibit unwanted excitation. Even under this circumstance, however, various implanted electrodes of the IMDs can corrode, electroplate, and/or otherwise degrade. Gradual degradation of such electrodes may eventually lead to total destruction thereof, formation of open circuit there around, and the like.

For example, when the electrodes implanted into the patient's right ventricle are shorted to the case, an output switch bridge or switch bank of the IMD 10 will be shorted out (HV energy switches generally fail in the on state) and deliver electric current directly into the surrounding cardiac tissues. Therefore, in the event of any high voltage application such as in cardioversion or defibrillation therapy, destroyed high-voltage switch bank and/or bridges thereof the high voltage charger may apply power of 4 watts directly to the surrounding tissues, thereby potentially placing the patient in a hazardous situation.

Accordingly, there is a need to provide IMDs with leakage current detection circuitry and circuitry to perform appropriate mitigating action when leakage current is detected.

### SUMMARY

In accordance with an embodiment, a medical device (external or implantable) is provided that comprises a power source, a charge storage member, a connector, a switch network, a controller and a leak detection module. The charge storage member is configured to receive and store energy from the power source. The connector is configured to be coupled to a lead to be implanted in a patient proximate to tissue of interest. The switch network is electrically disposed between the charge storage member and the connector. The switch network changes between open and closed states to disconnect and connect the charge storage member and the connector. The controller controls storage of energy in the charge storage member and delivery of stimulating pulses from the charge storage member to the lead coupled to the connector. The leak detection module obtains a leakage measurement by sensing at least one of i) a voltage potential of the charge storage member and ii) current flow from the charge storage member. The leak detection module compares the leakage measurement to a leakage threshold to determine when the leakage measurement satisfies the leakage threshold.

Optionally, the leakage detection module may further comprise a current sensor disposed between the charge storage member and the switch network. The current sensor may be disposed between the switch network and the connector. Optionally, the leakage detection module may comprise a voltage sensor disposed at the charge storage member to detect the voltage across the charge storage member. The current sensing member includes a resistor disposed in parallel with a diode to sense the current flow to or from the switch network. The current sensing circuitry may include at least two diodes disposed in parallel with the resistor, where the diodes are disposed to allow current flow in opposite directions. The resistor senses the current flow to or from the switch network. The diode or diodes bypass the resistor in order to allow for the delivery of shock current which is many orders of magnitude as leakage currents.

Optionally, the leakage threshold may constitute a preset current range. The leak detection module may identify a current leak when the current flow is outside of the preset current range. Optionally, the leakage threshold may constitute a preset voltage range and the leak detection module may identify a current leak when the voltage potential of the charge storage member is outside of the preset voltage range.

In accordance with an embodiment, the charge storage member is configured to receive the energy during a period of time in which at least a portion of the tissues of a heart are in a refractory condition. The controller controls the charge storage member to receive the energy during a charging period which is synchronized with at least one of an atrial event and a ventricular event of a heart. The controller may be configured to synchronize a charging period of the charge storage member with an R wave of the heart.

Optionally, the controller may be configured to sense at least one of the voltage potential and current flow continuously. Alternatively, the controller may be configured to sense at least one of the voltage potential and current flow intermittently. Alternatively, the controller may be configured to sense at least one of the voltage potential and current flow for a preset charging period.

Optionally, the controller may decouple the charge storage member from the power source upon detecting that the leakage measurement exceeds the leakage threshold. The controller may operationally decouple the electrode from the charge storage member upon detecting that the leakage measurement exceeds the leakage threshold. The controller is configured to issue at least one of a vibratory warning signal and an audible warning signal upon detecting that the leakage measurement exceeds the leakage threshold.

In accordance with an alternative embodiment, a method is provided for detecting energy leakage from a medical device (external or implantable). The method comprises initiating charge of a charge storage member in the medical device, and sensing at least one of i) a voltage potential of the charge storage member and ii) current flow from the charge storage member, to obtain a leakage measurement. The method further includes comparing the leakage measurement to a leakage threshold to determine when the leakage measurement exceeds the leakage threshold and identifying energy leakage when the leakage measurement satisfies the leakage threshold.

Optionally, the method may further comprise terminating charging of the charge storage member upon identifying energy leakage, and operationally decoupling the charge storage member from the tissue upon identifying energy leakage. The method may further comprise operationally decoupling the electrode from the charge storage member upon identifying energy leakage. The method may further comprise determining a refractory period of at least a portion of the tissue of interest and timing the initiating operation to begin charging the charge storage member during the refractory period.

Optionally, the method may comprise charging the charge storage member for a charging period ranging from about 20 msec to about 50 msec. before the sensing operation. The method synchronizes the initiating operation with at least one of an atrial event and a ventricular event of a heart. The method may monitor the current flowing in both directions between the tissue of interest and the charge storage member. The method may include obtaining at least one of an average voltage potential and average current flow over a preset period; and comparing at least one of the averaged voltage potential and averaged current flow to a preset voltage range and preset current range, respectively.

### BRIEF DESCRIPTION OF THE DRAWINGS

FIG. 1 is a simplified, partly cut-away view of an exemplary implantable medical device in electrical communication with at least three leads implanted into a patient's heart.

FIG. 2 is a functional block diagram of the IMD of FIG. 1.

FIG. 3 illustrates a schematic diagram of a switch network that may be located between the capacitors of a shocking circuit and the terminals of the connector in accordance with an embodiment.

FIG. 4 illustrates a circuit diagram of an exemplary charge storage member and leakage detection system in accordance with an embodiment

FIG. 5 illustrates a circuit diagram of an exemplary leakage detection system in accordance with an alternative embodiment.

FIG. 6 illustrates a current leakage detection process implemented by an IMD in accordance with an embodiment.

FIG. 7 illustrates an exemplary atrial cardiac event.

FIG. 8 illustrates an exemplary ventricular cardiac event.

FIG. 9 illustrates a charge timing process implemented in accordance with an embodiment.

FIG. 10 illustrates a post-leak assessment process performed after leakage confirmation in accordance with an embodiment.

### DETAILED DESCRIPTION

The following description is of a best mode presently contemplated for practicing the present invention. This description is not to be taken in a limiting sense but is made merely for the purpose of describing the general principles of the invention. In the description of the invention that follows, like numerals or reference designators will be used to refer to like parts or elements throughout. Although the following embodiments are described principally in the context of pacemaker/defibrillator unit capable of sensing and/or pacing pulse delivery, the medical system may be applied to other IMD structures and external medical devices. For example, embodiments may be implemented in an external defibrillator, external programmer and the like. For example, embodiments may be implemented in external defibrillators such as described in U.S. Pat. Nos. 7,272,441; 7,257,440 and 6,990,373. As further examples, embodiments may be implemented in leads for devices that suppress an individual's appetite, stimulate the patients nervous or muscular systems, stimulate the patient's brain functions, reduce or offset pain associated with chronic conditions and control motor skills for handicap individuals, and the like.

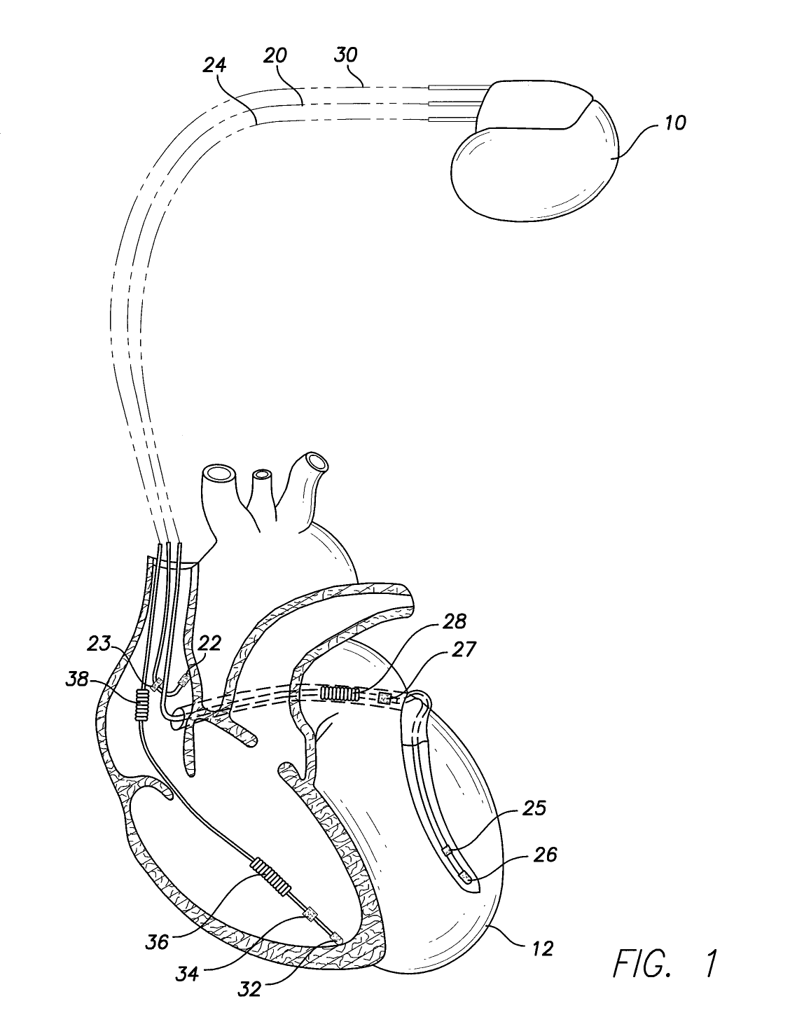

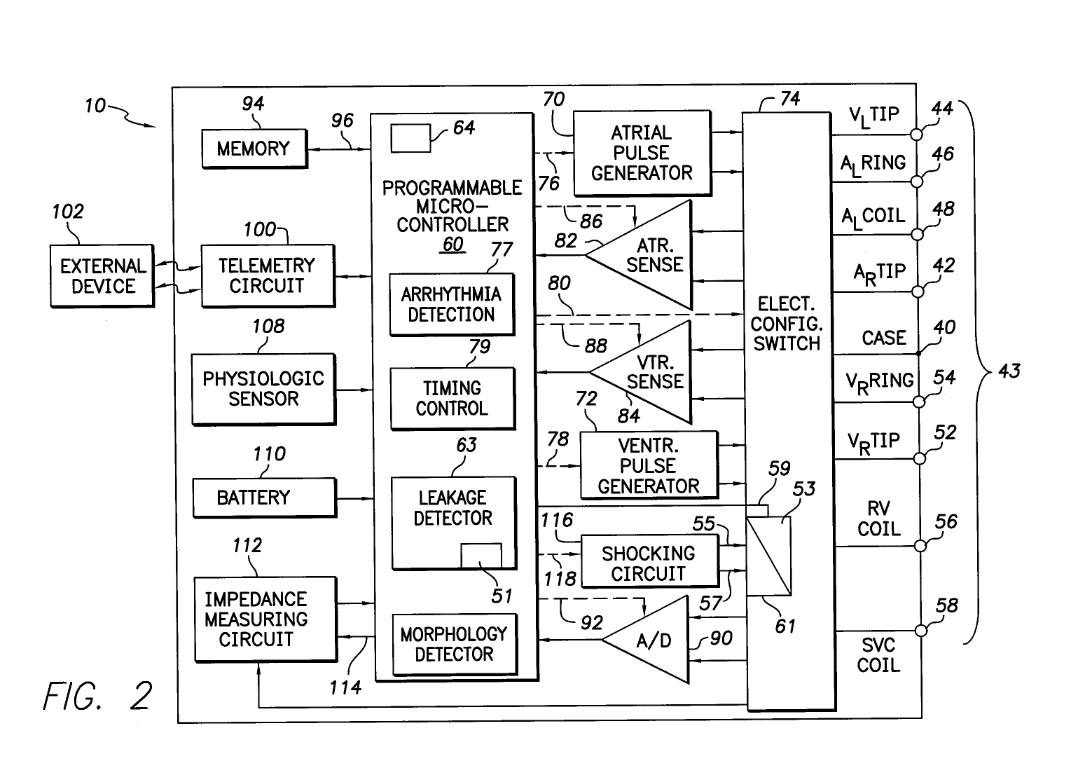

A cardiac stimulation device will thus be described in conjunction with FIGS. 1 and 2, in which the features included in this invention could be implemented. It is recognized, however, that numerous variations of such a device exist in which various methods included in the present invention can be implemented without deviating from the scope of the present invention.

FIG. 1 illustrates a IMD 10 in electrical communication with a patient's heart 12 by way of three leads 20, 24 and 30 suitable for delivering multi-chamber stimulation and/or shock therapy. To sense atrial cardiac signals and to provide right atrial chamber stimulation therapy, the device 10 is coupled to an implantable right atrial lead 20 including at least one atrial tip electrode 22 that typically is implanted in the patient's right atrial appendage. The right atrial lead 20 may also include an atrial ring electrode 23 to allow bipolar stimulation or sensing in combination with the atrial tip electrode 22.

To sense the left atrial and left ventricular cardiac signals and to provide left-chamber stimulation therapy, the IMD 10 is coupled to a “coronary sinus” lead 24 designed for placement in the “coronary sinus region” via the coronary sinus ostium in order to place a distal electrode adjacent to the left ventricle and additional electrode(s) adjacent to the left atrium. As used herein, the phrase “coronary sinus region” refers to the venous vasculature of the left ventricle, including any portion of the coronary sinus, great cardiac vein, left marginal vein, left posterior ventricular vein, middle cardiac vein, and/or small cardiac vein or any other cardiac vein accessible by the coronary sinus.

Accordingly, the coronary sinus lead 24 is designed to: receive atrial and/or ventricular cardiac signals; deliver left ventricular pacing therapy using at least one left ventricular tip electrode 26 for unipolar configurations or in combination with left ventricular ring electrode 25 for bipolar configurations; deliver left atrial pacing therapy using at least one left atrial ring electrode 27 as well as shocking therapy using at least one left atrial coil electrode 28.

The IMD 10 is also shown in electrical communication with the patient's heart 12 by way of an implantable right ventricular lead 30 including, in this embodiment, a right ventricular (RV) tip electrode 32, a right ventricular ring electrode 34, a right ventricular coil electrode 36, a superior vena cava (SVC) coil electrode 38, and so on. Typically, the right ventricular lead 30 is inserted transvenously into the heart 12 so as to place the right ventricular tip electrode 32 in the right ventricular apex such that the RV coil electrode 36 is positioned in the right ventricle and the SVC coil electrode 38 will be positioned in the right atrium and/or superior vena cava. Accordingly, the right ventricular lead 30 is capable of receiving cardiac signals, and delivering stimulation in the form of pacing and shock therapy to the right ventricle.

FIG. 2 illustrates a simplified block diagram of the multi-chamber IMD 10, which is capable of treating both fast arrhythmia and slow arrhythmia with stimulation therapy, including cardioversion, defibrillation, and pacing stimulation. While a particular multi-chamber device is shown, this is for illustration purposes only, and one of ordinary skill in the pertinent art could readily duplicate, eliminate or disable the appropriate circuitry in any desired combination to provide a device capable of treating the appropriate chamber(s) with cardioversion, defibrillation, and/or pacing stimulation.

The IMD 10 includes a housing 40 which is often referred to as “can,” “case,” or “case electrode,” and which may be programmably selected to act as the return electrode for all “unipolar” modes. The housing 40 may further be used as a return electrode alone or in combination with one or more of the coil electrodes 28, 36, or 38, for defibrillation shocking purposes. The housing 40 further includes a connector having a plurality of terminals 42, 43, 44, 45, 46, 48, 52, 54, 56, and 58 (shown schematically and, for convenience, the names of the electrodes to which they are connected are shown next to corresponding terminals). As such, in order to achieve right atrial sensing and stimulation, the connector includes at least one right atrial tip terminal (RA TIP) 42 adapted for connection to the atrial tip electrode 22. The connector may also include a right atrial ring terminal (RA RING) for connection to the right atrial ring electrode 23.

To achieve left chamber sensing, pacing, and/or shocking, such a connector includes a left ventricular tip terminal (LV TIP) 44, a left ventricular ring terminal (LV RING) 25, a left atrial ring terminal (LA RING) 46, and a left atrial shocking coil terminal (LA COIL) 48, that are adapted for connection to the left ventricular tip electrode 26, the left ventricular ring electrode 25, the left atrial ring electrode 27, and the left atrial coil electrode 28, respectively.

To support right ventricular sensing, pacing, and/or shocking, the connector may further include a right ventricular tip terminal (RV TIP) 52, a right ventricular ring terminal (RV RING) 54, a right ventricular shocking coil terminal (RV COIL) 56, and an SVC shocking coil terminal (SVC COIL) 58, which are adapted for connection to the right ventricular (RV) tip electrode 32, the RV ring electrode 34, the RV coil electrode 36, and the SVC coil electrode 38, respectively.

At the core of the IMD 10 is a programmable microcontroller 60 that controls the various modes of stimulation therapy. The microcontroller 60 typically includes a microprocessor, or equivalent control circuitry, designed specifically for controlling the delivery of stimulation therapy, and may include RAM or ROM memory, logic and timing circuitry, state machine circuitry, and/or I/O circuitry. Typically, the microcontroller 60 may have the ability to process or monitor various input signals (data) as controlled by a program code stored in a designated block of memory.

FIG. 2 illustrates an atrial pulse generator 70 and ventricular pulse generator 72 which generate stimulation pulses for delivery by the right atrial lead 20, the right ventricular lead 30, and/or the coronary sinus lead 24 via a switch 74. It is understood that, to provide the stimulation therapy in each of the four chambers of the heart, the atrial pulse generator 70 and the ventricular pulse generator 72 may include, e.g., dedicated pulse generators, independent pulse generators, multiplexed pulse generators, and/or shared pulse generators. The atrial pulse generator 70 and the ventricular pulse generator 72 are generally controlled by the microcontroller 60 via appropriate control signals 76 and 78, respectively, to trigger or inhibit the stimulation pulses.

The microcontroller 60 may further include timing control circuitry 79 which may be used to control timing of the stimulation pulses such as, e.g., pacing rate, atrio-ventricular (AV) delay, atrial interchamber (A-A) delay, and/or ventricular interchamber (V-V) delay. Such timing control circuitry 79 may also be used to keep track of the timing of refractory periods, noise detection windows, evoked response windows, alert intervals, marker channel timing, and so on.

The switch 74 includes a plurality of switches for connecting the desired electrodes to the appropriate I/O circuits, thereby providing complete electrode programmability. Accordingly, the switch 74, in response to a control signal 80 from the microcontroller 60, determines the polarity of the stimulation pulses (e.g., unipolar, bipolar, cross-chamber, and the like) by selectively closing the appropriate combination of switches. Atrial sensing circuits 82 and ventricular sensing circuits 84 may also be selectively coupled to the right atrial lead 20, coronary sinus lead 24, and the right ventricular lead 30 through the switch 74, for detecting the presence of cardiac activity in each of the four chambers of the heart.

Accordingly, the atrial sensing circuit 82 and the ventricular sensing circuit 84 may include dedicated sense amplifiers, multiplexed amplifiers or shared amplifiers. The switch 74 determines the “sensing polarity” of the cardiac signal by selectively closing the appropriate switches. In this way, the clinician may program the sensing polarity independent of the stimulation polarity.

Each of the atrial and ventricular sensing circuits 82, 84 preferably employs one or more low power, precision amplifiers with programmable gain, automatic gain or sensitivity control, band-pass filtering, and threshold detection circuit, to selectively sense the cardiac signal of interest. The automatic sensitivity control enables the IMD 10 to deal effectively with the difficult problem of sensing the low amplitude signal characteristics of atrial or ventricular fibrillation.

The outputs of the atrial sensing circuit 82 and ventricular sensing circuits 84 may be connected to the microcontroller 60 for triggering or inhibiting the atrial and ventricular pulse generators 70 and 72, respectively, in a demand fashion, in response to the absence or presence of cardiac activity, respectively, in the appropriate chambers of the heart. The atrial and ventricular sensing circuits 82 and 84, in turn, may receive control signals over signal lines 86 and 88 from the microcontroller 60, for controlling the gain, threshold, polarization charge removal circuitry, and the timing of any blocking circuitry coupled to the inputs of the atrial and ventricular sensing circuits 82 and 84.

For arrhythmia detection, the IMD 10 includes an arrhythmia detector 77 that utilizes the atrial and ventricular sensing circuits 82 and 84 to sense cardiac signals, for determining whether a rhythm may be physiologic or pathologic. As used herein, “sensing” generally refers to the process of noting an electrical signal, while “detection” generally refers to the step of confirming the sensed electrical signal as the signal being sought by the detector. As an example, “detection” applies to the detection of both proper rhythms (i.e., “P wave” or “R wave”) as well as improper disrhythmias including arrhythmia and bradycardia (e.g., detection of the absence of a proper rhythm).

The timing intervals between sensed events (e.g., P-waves, R-waves, and depolarization signals associated with fibrillation which are sometimes referred to as “F-waves” or “Fib-waves”) are then classified by the arrhythmia detector 77 by comparing them to a predefined rate zone limit (e.g., bradycardia, normal, low rate ventricular tachycardia, high rate ventricular tachycardia, fibrillation rate zones, and so on) and various other characteristics (e.g., sudden onset, stability, physiologic sensors, morphology, and so on), in order to determine the type of remedial therapy required (e.g., bradycardia pacing, anti-tachycardia stimulation, cardioversion shocks or defibrillation shocks, collectively referred to as “tiered therapy”).

Cardiac signals are also applied to the inputs of a data acquisition system 90 which is depicted as an analog-to-digital (ND) converter for simplicity of illustration. The data acquisition system 90 is configured to acquire intracardiac electrogram (e.g. IEGM) signals, convert the raw analog data into digital signals, and store the digital signals for later processing and/or telemetric transmission to an external device 102. The data acquisition system 90 may be coupled to the right atrial lead 20, the coronary sinus lead 24, and the right ventricular lead 30 through the switch 74. The data acquisition system 90 may sample the cardiac signals across any pair of desired electrodes. The data acquisition system 90 may be coupled to the microcontroller 60 and/or another detection circuitry and controlled by signal 92, for detecting an evoked response from the heart 12 in response to an applied stimulus, thereby aiding in the detection of “capture.” Detecting the evoked response during the detection window may indicate that capture has occurred.

The microcontroller 60 may further be coupled to a memory 94 by a suitable data/address bus 96, wherein the programmable operating parameters used by the microcontroller 60 are stored and modified, as required, so as to customize the operation of the IMD 10 to suit the needs of particular patients. Such operating parameters may define, e.g., stimulation pulse amplitude, pulse duration, polarity of electrodes, rate, sensitivity, automatic features, arrhythmia detection criteria, and/or the amplitude, shape of waves, and/or vector of each stimulation pulse to be delivered to the patient's heart 12 within each respective tier of therapy.

The IMD 10 may additionally include a power source that may be illustrated as a battery 110 for providing operating power to all the circuits of FIG. 2. For the IMD 10 employing shocking therapy, the battery 110 must be capable of operating at low current drains for long periods of time, preferably less than 10 uA, and also be capable of providing high-current pulses when the patient requires a shock pulse, preferably in excess of 2 A, at voltages above 2 V, for periods of 10 seconds or more. The battery 110 preferably has a predictable discharge characteristic such that elective replacement time can be detected. A physiologic sensor 108 detects motion of the IMD and thus, patient to determine an amount of activity.

A patient warning signal generator 64 may be included in the microcontroller 60 so that a patient or operator may be alerted to a condition requiring medical attention. A condition warranting a patient alarm may be related to operation of the IMD 10 or may be related to a detected patient condition. For example, patient warning systems have been proposed for alerting a patient to a detected tachycardia and impending stimulation therapy delivery. In accordance with one exemplary embodiment, the patient warning generator 64 may be used to alert the patient or operator to current leakage detection as will be described later. Exemplary patient warning signals include a twitch sensation caused by delivery of a stimulation pulse or burst of pulses delivered to excitable tissue, or an audible warning sound, or a vibratory warning signal.

The IMD 10 includes an impedance measuring circuit 112 which is enabled by the microcontroller 60 by control signal 114. The known uses for an impedance measuring circuit 112 include, but are not limited to, lead impedance surveillance during the acute and chronic phases for proper lead positioning or dislodgement; detecting operable electrodes and automatically switching to an operable pair in case dislodgement should occur; measuring respiration or minute ventilation; measuring thoracic impedance for determining shock thresholds; detecting when the device has been implanted; measuring stroke volume; detecting opening of heart valves, and so on. The impedance measuring circuit 112 is advantageously coupled to the switch 74 so that any desired electrode may be used.

The IMD 10 may be used as an implantable cardioverter defibrillator (ICD) device by detecting the occurrence of an arrhythmia, and automatically applying an appropriate electrical stimulation or shock therapy to the heart aimed at terminating the detected arrhythmia. To this end, the microcontroller 60 further controls a shocking circuit 116 by way of a control line 118. The shocking circuit 116 includes charge storage members, such as one or more capacitors. The charge storage members are charged by the battery 110 before delivering stimulating energy such as high voltage shocks. The charge storage members deliver the stimulating energy over positive and negative lines 55 and 57. The switch 74 includes a switch network 61 that is electrically disposed between the positive and negative lines 55 and 57, and the appropriate terminals of the connector 43. The switch network 61 changes between open and closed states to disconnect and connect the charge storage members and the connector 43.

A leak detection module 63 is provided at the controller 60 to obtain leakage measurements. The leakage measurements are obtained by a leak sensing circuit 53 located proximate the switch network 61. The leak sensing circuit 53 may be located upstream or down stream of the switch network 61 depending in part on the type of leak detection to be performed. The leak sensing circuit 53 may sense a voltage potential across the charge storage member in the shocking circuit 116. Alternatively, or in addition, the leak sensing circuitry 53 may sense current flow through the switch network 61 and thus from the charge storage member. The leak sensing circuitry 53 provides a leakage measurement (denoted at line 59) to the leakage detection module 63. The leak sensing circuitry 53 may represent a voltage sensor, a current sensor a power sensor, a combination thereof and the like.

Optionally, to detect leakage, the controller 60 may interrupt a charging operation and measure a voltage potential across the capacitors in the shocking circuit 116 over control line 118. The controller 60 passes this voltage measurement to the leakage detection module 63 which determines whether the voltage potential across the capacitors of the shocking circuit 116 corresponds to an expected charge pattern. Optionally, the voltage potential may be measured without interrupting a charging operation of the capacitors.

The controller 60 manages operation of the leak sensing circuitry 53 to obtain sensor reads (e.g. sense) of at least one of the voltage potential and current flow continuously and at all times throughout operation. Alternatively, the controller 60 manages operation of the leak sensing circuitry 53 to obtain sensor reads (e.g. sense) of at least one of the voltage potential and current flow intermittently. As a further option, the controller 60 may cause sensing to occur periodically after a preset charging period.

The leak detection module 63 compares the leakage measurement 59 to a leakage threshold 51 to determine when the leakage measurement 59 exceeds the leakage threshold 51. The leakage threshold may be pre-programmed and/or programmable by a physician using a programmer and the like. The leakage threshold may constitute a preset voltage range with upper and lower limits. The leak detection module 63 may identify current leakage when the voltage potential of the charge storage member is outside of the preset voltage range.

Optionally, the controller 60 may control an initial charge period for the shocking circuit 116 such that the charge storage member initially only receives the energy from the battery 110 during a period of time in which a desired portion of the tissue of the heart is in a refractory state. For example, the charging period may be synchronized to occur only during the atrial refractory period. Alternatively, the charging period may be synchronized to occur only during the ventricular refractory period. As one example of a manner to synchronize the charging period to a desired refractory period, the start time at which a charging period is initiated may be set a predetermined number of milliseconds after the occurrence of an R-wave.

The controller 60 may attempt to mitigate or correct for leakage. For example, the controller 60 may decouple the charge storage member of the charging circuit 116 from the battery 110 when the leakage detection module 63 detects a leakage measurement that satisfies the leakage threshold (e.g. the leakage measurement represents an amount of current that exceeds a maximum acceptable amount of leakage current, or the leakage measurement is a voltage potential that is beyond a threshold voltage that is expected across the HV capacitors). The decoupling may occur by disconnecting charge storage capacitors entirely or by disabling a portion of firmware/software that initiates a charging operation. When an electrode is operationally coupled to the charge storage member and the tissue, the controller 60 operationally decouples the electrode from the charge storage member when the leakage measurement satisfies the leakage threshold. The controller may also issue at least one of a vibratory warning signal and an audible warning signal, from the patient warning system 64, upon detecting that the leakage measurement satisfied the leakage threshold (e.g. exceeds a maximum or falls below a minimum).

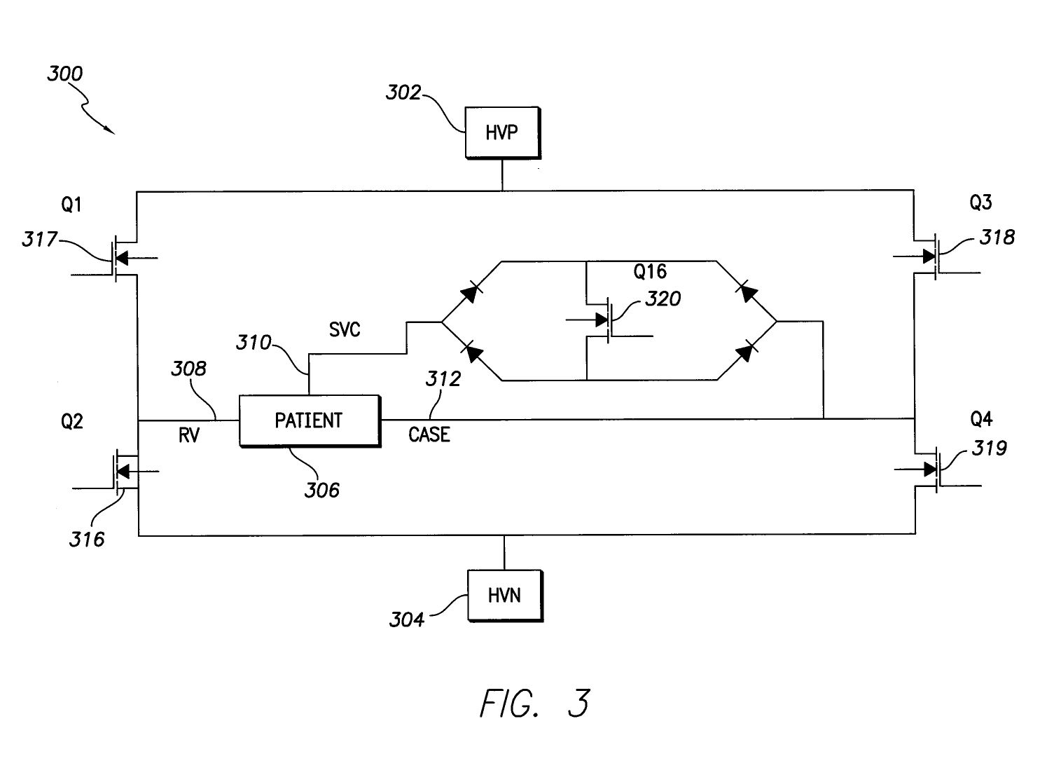

FIG. 3 illustrates a schematic diagram of a switch network 300 (such as network 61) that may be located between the capacitors of a shocking circuit 116 and the terminals of the connector 43. The switch network 300 includes high voltage positive and negative nodes 302 and 304 that are connected to the positive and negative terminals of one or more energy storage capacitors in the shocking circuit 116. A group of switching transistors 316-320 is joined in the H-bridge architecture with the positive and negative nodes 302 and 304. The transistors 316-320 are controlled by the controller 60 (FIG. 2) to change between open circuit and closed circuit states. The transistors 316-320 connect and disconnect the positive and negative nodes 302 and 304 to desired combinations of electrodes, such as an RV electrode 308, a CAN electrode 312 and a SVC electrode 310, which are located proximate tissue of interest 306 in a patient. The transistors 316-320 in the example of FIG. 3 represent IGBT transistors.

When the transistors 316-320 fail, these IGBT transistors usually fail to a short state in which the source and drain are shorted together when too much power must be dissipated by the switch network 300. As noted above, high power dissipation may be introduced when the lead, or one or more of the electrodes (e.g. 308, 310, 312), fails in a shorted state. High power dissipation may also be necessary when an external defibrillator is utilized on the patient which then causes a dielectric breakdown. When high power is introduced across the switch network, it may destroy two or more of the transistors 316-320. When opposing transistors 317 and 319 fail, this may create a closed circuit between the positive and negative nodes 302 and 304 through transistors 317 and 319, and through electrodes 308 and 312 to the tissue 306 of the patient. Alternatively, when opposing transistors 316 and 318 fail, this may create a closed circuit between the positive and negative nodes 302 and 304 through transistors 316 and 318, and through electrodes 308 and 312 to the tissue of the patient. Thus, the patient may be exposed to leakage current that may induce fibrillation or other reactions when the IMD is attempting to charge the capacitors. The failure of the switch network 300 may create a complete short or a near short connection (e.g., a connection exhibiting low impedance such as less than 20 ohms) between the high voltage charge circuit and the terminals of the connector 43 (FIG. 2).

When same-side transistors 316 and 317 fail, this may create a closed circuit between the positive and negative nodes 302 and 304 through transistors 316 and 317. While a same-side transistor failure may not introduce spurious current into the patient, a same-side transistor failure may prevent the capacitors from charging and drain the battery unduly earlier.

In accordance with at least one embodiment, the IMD 10 measures the voltage across the positive and negative nodes 302 and 304 of the switch network 300. The IMD 10 may measure the voltage across the positive and negative terminals of one or more capacitors in the charge storage member (in shocking circuit. For example, with reference to FIG. 2, the IMD 10 may measure the voltage potential across the positive and negative lines 55 and 57.

Various components of IMD 10 may be degraded, shorted, and leak electric energy to the surrounding cardiac tissues. Embodiments of the present invention provide systems and associated methods to detect the leaking current, to issue visual or audible warning signals to the patient or medical staff, and to take remedial actions in order to minimize or prevent further leakage there from. As an example, a leakage detection system and method may be provided for detecting current flow leaking from the IMD 10 by measuring the voltages across pre-selected components of the IMD 10, rates of charge buildup in a capacitor and/or the rate of charge depletion of the IMD 10 capacitors after a predetermined waiting period, current flows through various components of the IMD 10, and similar other indicators. Upon detecting the leakage, The IMD 10 may be configured to issue various vibratory and/or audible warning signals and to take remedial actions to minimize further leakage of energy or current by, e.g., eliminating the leakage components from the network, terminating the charging process of the charge generators or capacitors, and terminating the operation of at least a portion of the IMD 10 until proper remedial action is taken.

Various embodiments of a leakage detection and warning system will now be described. It is recognized, however, that numerous variations of such systems and methods exist without deviating from the scope of the present invention. As noted above in connection with FIG. 3, the controller 60 may be configured to monitor voltage or voltage change over time in at least one location of the IMD 10 and to detect current leakage based thereon.

While embodiments described herein utilize IGBT transistors in an H-bridge configuration, optionally embodiments of the present invention may be implemented utilizing other types of circuits and other switch bridge configurations. For example, an H-bridge switch could be implemented utilizing field effect transistors (FETs), silicon controlled rectifiers (SCRs), unijunction transistors (UJTs), bipolar junction transistors (BJTs), relays or any combination thereof. Optionally, vacuum tube switches, such as triodes, tetrodes, pentodes, etc., could be used to form the switch network. Optionally, the switch network may be implemented utilizing a network of circuits in a configuration that differs from an H-bridge.

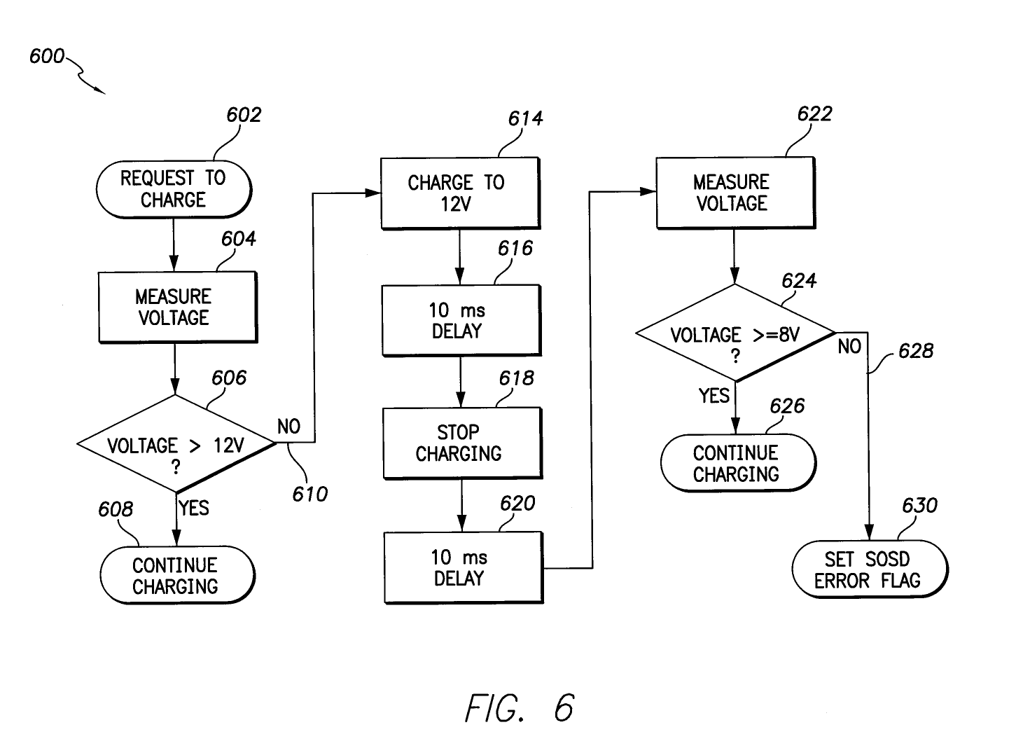

FIG. 6 illustrates a current leakage detection process implemented by the IMD 10 in accordance with an embodiment of the present invention. The leakage detection process 600 may be implemented in connection with a voltage measurement circuit that measures the voltage across the charge storage member and/or the switch network. The process 600 begins when the controller 60 requests to initiate a charge operation at 602. At 604, the controller 60 performs an initial voltage measurement at the beginning of the charging operation. For example, the initial voltage measurement may be across lines 55 and 57 (FIG. 2), or nodes 302 and 304 (FIG. 3). Next at 606 the leakage detection module 63 determines whether the initial voltage measurement exceeds an initial charge threshold. In the example of FIG. 6, the initial measurement represents a voltage and the initial charge threshold a voltage, such as 12 volts. Optionally, the initial measurement may represent a current measurement and the initial charge threshold represents a current threshold. When the initial charge measurement exceeds the initial charge threshold, this indicates that the charge storage member is not loosing charge, and instead already has an amount of stored energy that is indicative of a non-leak condition. For example, it may have been determined that when energy leaks from the charge storage member the level of charge on the charge storage member does not rise to the initial charge threshold (e.g. 12V). When the voltage measured on the charge storage member exceeds 12 volts this is an indication that the charge storage member is in an expected, fault-free condition. Hence, no further leakage detection is warranted. Hence, when, at the beginning of a charge request, the charge storage member already has a voltage potential of over 12V, flow moves to 608 where charging is continued.

However, when the initial charge measurement is less than the initial charge threshold (e.g. less than 12 volts), then this is an indication that the charge storage member may potentially not be operating in an expected, fault-free condition. Therefore, further testing is warranted. Hence, flow moves along 610 to 614. At 614, the controller 60 begins to attempt to charge the charge storage member to a predetermined level, which may correspond to the initial charge threshold (e.g. 12 volts). At 616, a delay is introduced (e.g. 10 msec.) during which charge is applied to the charge storage member. The delay may be for a predetermined or programmable period of time. Alternatively, the delay may be for a time determined by the controller 60 based on the condition of the battery 110. After the delay at 616, the charging operation is stopped at 618. At 620, another delay is introduced (e.g. 10 msec). This second post-charge delay is set to afford the charge storage member an opportunity to hold or loose its charge. When the IMD 10 is in a fault-free condition, the charge storage member will hold the charge.

At 622, a leakage measurement (e.g. a voltage measurement) is obtained by the leakage detection module 63. For example, the leakage measurement may represent a voltage potential across the lines 55 and 57, across the terminals of the charge storage member and/or across the nodes 302 and 304 of the switch network 300. At 624, the leakage measurement is compared with a leakage threshold (e.g. 8V). When the leakage measurement exceeds the leakage threshold, it is determined that the charge storage member is holding the charge in an expected fault-free manner. Thus, flow moves to 626. However, when the leakage measurement does not exceed the leakage threshold, it is determined that the charge storage member has lost a portion of the prior charge applied thereto. Hence, a current leakage condition exits and flow moves along 628 to 630 where a leakage identification error flag is set. Once the flag is set at 630 various actions may be taken as described throughout.

The process 600 may be repeated once every time the IMD 10 initiates a charging operation. Alternatively, the process 600 may be repeated after a preset number of charging operations. Alternatively, the process 600 may be repeated periodically (e.g. one per day, once per week, etc.). The process 600 monitors the rate of rise of the voltage on the charge storage member. The IMD 10 measures the voltage after a short initial-charge interval, such as 20-50 msec., which occurs at 614 in FIG. 6. During the initial-charge interval at 614 the rise in the voltage (charge pattern) of a normally functioning IMD 10 should be approximately 20-50V. The rise in voltage of a normal charge pattern may be more or less depending upon the capacitance of the charge storage members and the charging current that the battery is able to deliver. If the actual charge pattern has a voltage rise that is slower than the expected charge pattern, then the IMD 10 may be experiencing current leakage. The process 600 of FIG. 6 may be implemented in the firmware of the IMD 10 without the addition of new hardware components.

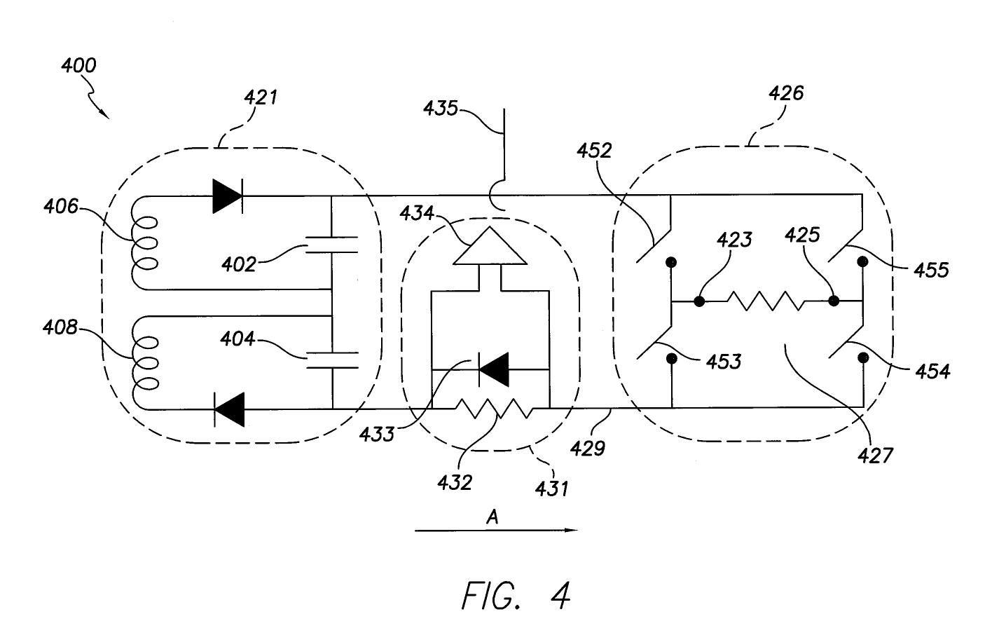

FIG. 4 illustrates a circuit diagram of an exemplary charge storage member and leakage detection system 400 to detect current leakage from the IMD 10 to surrounding excitable cardiac tissues by sensing voltage in a charge storage member. The system 400 generally includes a charge (or energy) storage member 421, a switch bank 426, a load 427 and a current sensing circuit 431, all of which are configured to operationally connect to each other under the general control of the controller 60 and under partial control of the leakage detection module 63 of FIG. 2.

The charge storage member 421 includes multiple capacitors 402 and 404 that are chargeable through transformers 406 and 408 by the battery 110. The switch bank 426 is generally configured to form a H-shaped bridge or a H-bridge, in which four switches 452-455 are disposed along legs of the bridge (or switch bank) 426. An external load 427 is illustrated. The external load 427 represents the tissue of interest (e.g., the heart or another organ), proximate to which electrodes are positioned as illustrated in FIGS. 1 and 3. The switch bank 426 connects and disconnects the charge storage member 421 to connector terminals 423 and 425. The connector terminals 423 and 425 are joined to one or more electrodes. For example, the connector terminals 423 and 425 may represent any of the terminals 40-58 in connector 43 of FIG. 2.

The current sensor circuit 431 is disposed along line 429 between the charge storage member 421 and the switch network 426. The current sensor circuit 431 includes a resistive load 432 located along the line 429. The resistive load 432 is provided in series with a positive or negative node of the switch network 426 which, when closed, becomes coupled to one of the connector terminals 423, 425. The resistive load 432 forms a current sensing resistor. A relatively low voltage potential is formed across the resistive load 432 when leakage current flows in line 429.

A diode 433 is connected in parallel with the resistive load 432. When the diode 433 is forward biased, the diode 433 has a maximum forward voltage drop of less than 2 Volts depending on the amount of current flow. The forward biased diode then bypasses the resistive load 432 when current flows in the direction of arrow A. Thus, the diode 433 limits the amount of energy wasted by the resistive load 432 to avoid any undue impact on the delivered energy to the patient. A sensing circuitry 434 detects the voltages at the input and output nodes of the resistive load 432. Current flow along line 429 is unidirectional and thus only a single diode 433 is utilized. The diode 433 is rated to withstand the full current capability of the IMD 10 such as a 50 Amp shock. The resistive load 432 may be chosen to sense leakage current above a value that will cause harm to the tissue of interest. For example, a shocking lead may have an area of 4 cm². When using the criteria of 1.5 mA/cm², then the resistive load 432 will be chosen to detect a gross leakage of 6 milliAmps. For example, a resistive load 432 of 100 ohms may be used. The current sensor circuit 431 may be only turned on during a high voltage charging operation.

In one embodiment, the sensing circuitry 434 may be a comparator that produces a difference signal (denoted as signal 435). When the sensing circuitry 434 is a comparator, the signal 435 corresponds to the difference between the voltage across the resistive load 432 and a preset voltage that corresponds to the maximum allowable leakage current, both are inputs to the sensing circuitry 434. When the sensing element 434 is a comparator, the comparator is configured such that the signal 435 changes between a logical high state and a logical low state. For example, the signal 435 may have the logical low state when no current flows in line 429. The signal 435 switches to the logical high state when current begins to flow in line 429 in the direction of arrow A. The signal 435 is provided to the leakage detection module 63 (FIG. 3). The leakage detection module 63 measures the signal 435 to determine whether signal 435 is in a logical high state or in a logical low state. For example, the leakage threshold may be satisfied by (e.g. correspond to) one of the logical high and low states. The leakage detection module 63 monitors the signal 435 to identify when current is flowing. Current should be flowing during delivery of therapy, but not between therapies. For example, when no therapy is being delivered (therapy-free), the controller 60 instructs the switch bank 426 to change to an open state and disconnect the connector terminals 423, 425 from the charge storage member 421. When in a therapy-free phase, no current should be flowing through line 429. During the therapy-free phase, when the leakage detection module 63 identifies current flow, this is an indication that current leakage may be occurring.

In another embodiment, the sensing circuitry 434 may be an analog to digital (A/D) converter that produces a digital data value, as the signal 435 that corresponds to the voltage potential across the resistive load 432. The digital data value, as signal 435, is supplied to the leakage detection module 63 in the controller 60. The leakage detection module 63 analyzes the digital data value to identify the level of leakage current.

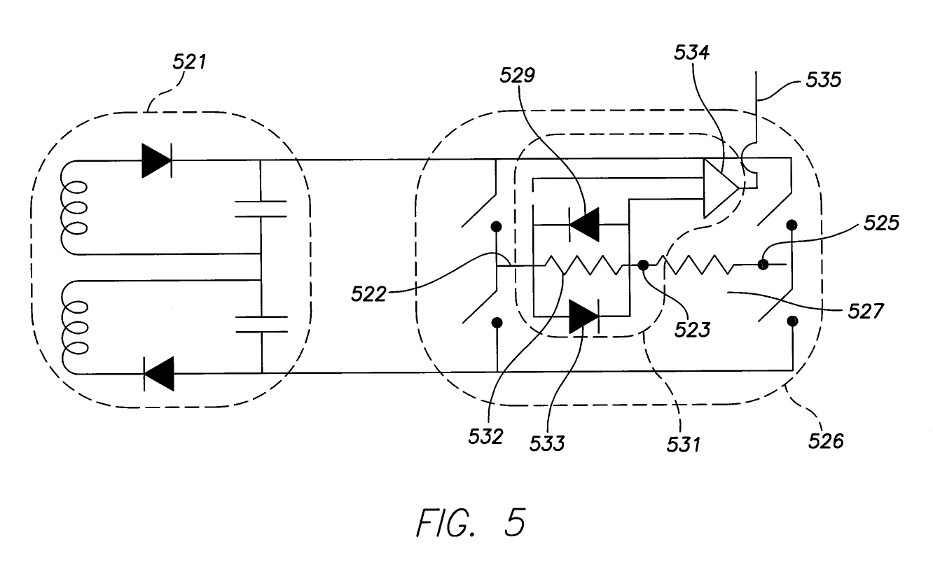

FIG. 5 illustrates a circuit diagram of an exemplary leakage detection system 500 formed in accordance with an alternative embodiment. The leakage detection system 500 detects current leakage from the IMD 10 to surrounding excitable cardiac tissues by sensing current flow to the lead. The leakage detection system 500 generally includes a charge (or energy) storage member 521, a switch bank 526, and a load 527, all of which are configured to operationally connect to each other under the control of the controller 60 and in part under the control of the leakage detection module 63 of FIG. 2.

The external load 527 represents the tissue of interest (e.g., the heart or another organ), proximate to which electrodes are positioned as illustrated in FIGS. 1 and 3. The switch bank 526 connects and disconnects the charge storage member 521 to connector terminals 523 and 525. The connector terminals 523 and 525 are joined to one or more electrodes.

A current sensor circuit 531 is disposed along line 522 between the switch network 526 and the connector terminal 523. Optionally, the current sensor circuit 531 could be disposed between the switch network 526 and the connector terminal 525. The current sensor circuit 531 includes a resistive load 532 located along the line 522. The resistive load 532 is provided in series with a positive or negative node of the switch network 526 which, when closed, becomes coupled to the connector terminal 523. The resistive load 532 forms a current sensing resistor. A relatively low voltage potential is formed across the resistive load 532 when current flows in line 522.

Diodes 529 and 533 are connected in parallel with the resistive load 532. The diodes 529 and 533 are oriented in opposite directions such that diode 529 is reverse bias (in an open circuit state) when diode 533 is forward bias (in a closed circuit state). In reverse, diode 529 is forward bias (in a closed circuit state) when diode 533 is reverse bias (in an open circuit state). When either of the diodes 529 or 533 is forward biased, the forward bias diode 529 or 533 has a maximum forward voltage drop of less than 2 Volts depending on the amount of current flow. The forward biased diode, either 529 or 533, then bypasses the resistive load 532 when current flows in either direction through line 522. The diodes 529 and 433 limit the amount of energy wasted by the resistive load 532 to avoid any undue impact on the delivered energy to the patient.

A sensing circuitry 534 detects the voltage across the resistive load 532 and outputs a signal 535. The sensing circuitry 534 may represent a comparator or an analog to digital converter. The signal 535 switches between logical high and low states when the sensing element 534 is a comparator. The signal 535 represents a digital data value of a measured voltage potential or current flow when the sensing circuitry 534 is an A/D converter.

The leakage detection module 63 is also configured to manipulate at least one of the switches 452-455 of the switch bank 426, thereby manipulating operational configurations of various terminals such as terminals 40, 42-46, 48, 52, 54, 56, 58 of FIG. 2. Once the charge storage member 421 is charged for a preset charging period, the leakage detection module 63 senses the voltage of the charge storage member 421 and, when desirable, calculates the rate of change of the voltage. The leakage detection module 63 compares the sensed voltage and the calculated rate of change in the voltage to a preset threshold voltage and a preset threshold rate of change (e.g., rise), respectively. When the sensed voltage of the charge storage member 421 is found to be less than the preset threshold voltage and/or when the calculated rate of change in the voltage falls below the preset threshold rate, the leakage detection module 63 identifies such behavior as a current leakage.

Optionally, the leakage detection module 63 may suspend the charger for a short interval (approximately <100 ms) and monitor the voltage decay on the charge storage members 421. If the voltage at the end of the short interval has decayed below a certain threshold, then the behavior is identified to indicate current leakage from the charge storage members 421.

In accordance with an embodiment, the controller 60 is programmed to charge the charge storage member 421 for a preset charging period, which is generally less than, about a hundred msec and, more particularly, less than about 50 msec. However, the charging period may vary according to various physiological or pathological conditions of the patient's heart, and electrical characteristics of the charge storage member 421 such as its capacitance. At the end of the charging period, normally functioning, non-leaking charge storage member 421 is charged generally to a set range of, for example, 10 volts to 50 volts, corresponding to a range of the rate of increase in voltage from about 0-12 V in about 3 msec. Such a rate may generally be obtained as the rate averaged over the beginning of the charging period. The leakage detection module 63 then compares the sensed voltage to the preset threshold voltage or compares the calculated rate of increase or decrease in voltage with the preset threshold rate. The leakage detection module 63 is generally configured to identify current leakage upon detecting the sensed voltage that is less than the preset voltage and/or upon detecting the calculated rate that fails to reach the preset rate. It is understood that the preset standard voltage of the charge storage member 421 as well as the preset standard rate of increase in the voltage can vary with each IMD. Advantageously, the foregoing exemplary leakage detection system 400 can be algorithmically implemented in pre-existing IMDs without adding new hardware. That is, the foregoing embodiment can readily be practiced in pre-existing IMDs by reprogramming their controllers to compare the sensed voltages to the preset standard values and/or to compare the calculated rates of change in the voltage of their pulse generators or other capacitors to the preset standard rates.

When an IMD 10 is operating in a current leakage condition, current flows from the IMD 10 to the tissue of interest whenever the IMD 10 begins to charge the charge storage member. In certain instances, the tissue of interest may be the heart and the lead may be located in the right atrium or right ventricle. When left unmanaged, the leakage current may be delivered to the atrium or ventricle at a time in the cardiac cycle during which the atrium and/ventricle are responsive to electrical stimulation. When sufficient leakage current is delivered at a time when the atrium and/ventricle are responsive to electrical stimulation, then the leakage current may capture the myocardium similar to a pacing pulse. It may be desirable to prevent the leakage current from interfering with the normal sinus rhythm of the heart.

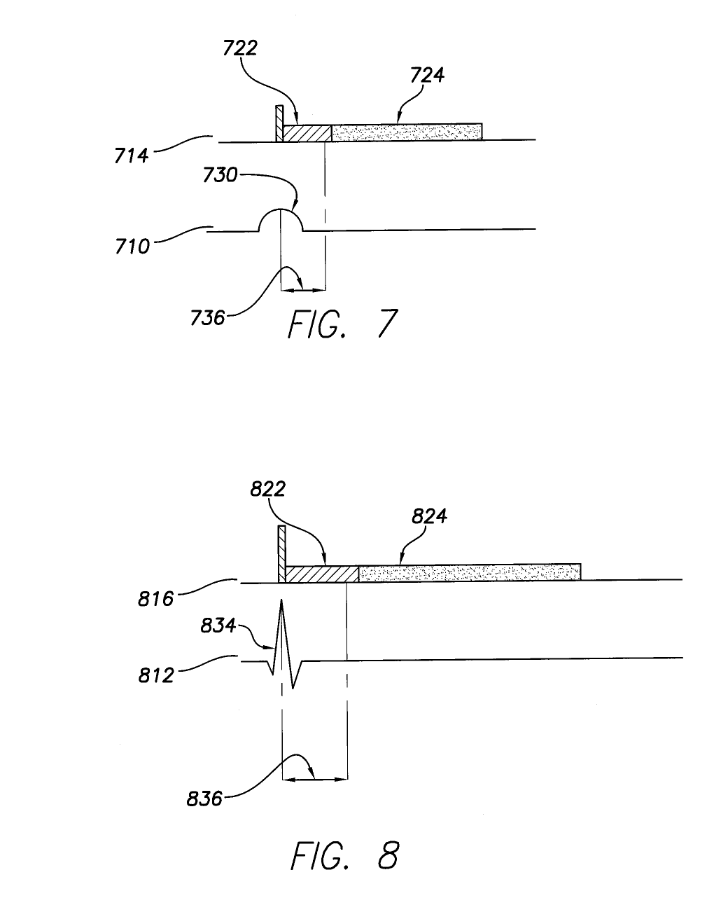

FIG. 7 illustrates an exemplary atrial cardiac event. FIG. 7 illustrates an atrial electrocardiogram (EGM) 710 aligned in time with an atrium channel 714. When a P-wave 730 occurs, the atrium enters an absolute atrium refractory period 722, followed by a relative atrium refractory period 724. During the absolute refractory period 722, the atrium is not sensitive to electrical stimulation. The controller 60 times the start and end times for the initial charge period to align with the refractory period 722. The controller 60 defines an atrial charge window 736 that extends from the P-wave for a period of time that is no longer than the absolute refractory period 722. By aligning the atrial charge window 736 with the absolute refractory period 722, the controller 60 avoids introducing charge into the charge storage member during a time period when the atrium is sensitive to electrical stimulation. If the atrium is refractory when the IMD 10 starts to charge the charge storage member, then any leakage current that might escape does not interfere with the atrium normal sinus rhythm.

FIG. 8 illustrates an exemplary ventricular cardiac event. FIG. 8 illustrates a ventricular electrocardiogram (EGM) 812 aligned in time with a ventricular channel 816. When an R-wave 834 occurs, the ventricle enters an absolute ventricular refractory period 822, followed by a relative ventricular refractory period 824. During the absolute refractory period 822, the ventricle is not sensitive to electrical stimulation. The controller 60 times the start and end times for the initial charge period to align with the refractory period 822. The controller 60 defines a ventricular charge window 836 that extends from the R-wave for a period of time that is no longer than the absolute refractory period 822. By aligning the ventricular charge window 836 with the absolute refractory period 822, the controller 60 avoids introducing charge into the charge storage member during a time period when the ventricle is sensitive to electrical stimulation. If the ventricle is refractory when the IMD 10 starts to charge the charge storage member, then any leakage current that might escape does not interfere with the ventricles normal sinus rhythm.

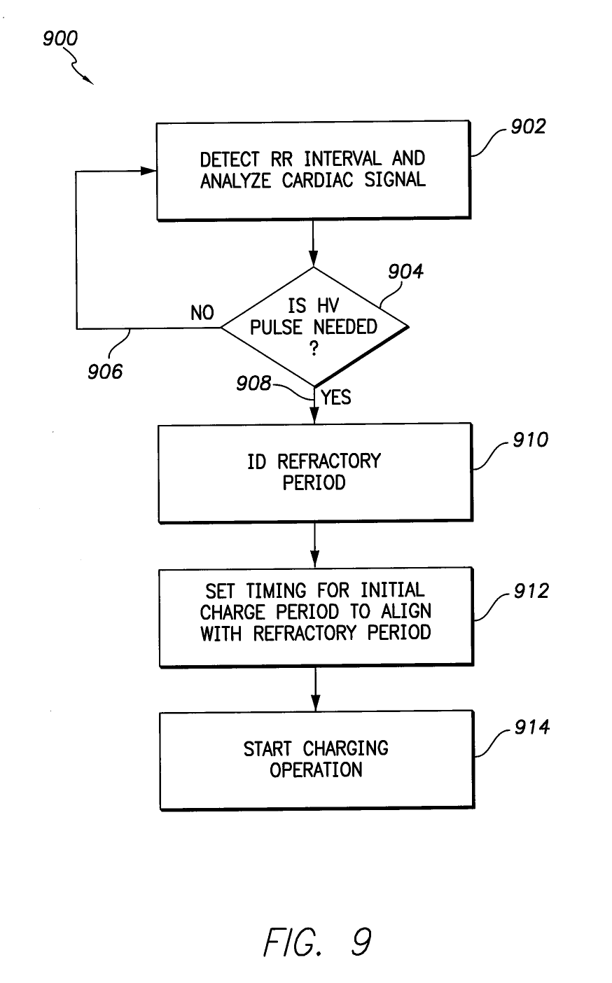

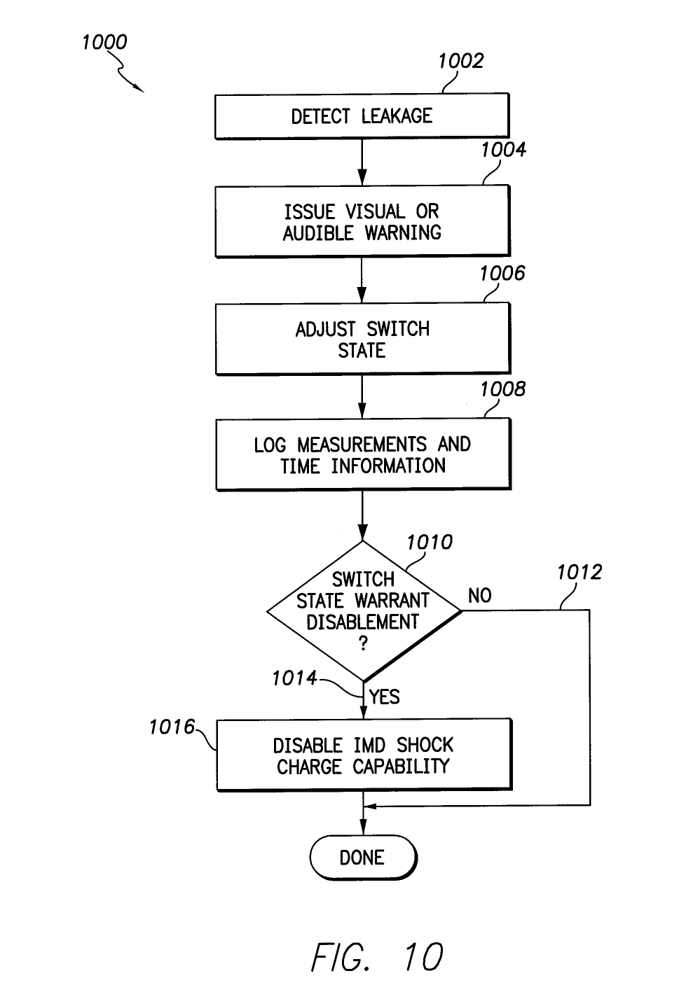

FIG. 9 illustrates a charge timing process 900 implemented in accordance with an embodiment to time the initial charge period to overlap the atrial or ventricular refractory period 722 or 822. At 902, the IMD 10 detects and analyzes cardiac signals. Among other things, the IMD 10 detects the R-R interval and analyzes the cardiac signals to determine if an arrhythmia is present. If certain arrhythmias are present, then the IMD 10 will determine that a high voltage shock should be delivered. At 904, the IMD 10 determines whether the cardiac signal indicates that a HV shock is needed. If no shock is needed, flow returns along path 906. When an HV shock is needed, flow moves along 908. At 910, the controller 60 identifies the start time of the refractory period of interest. For example, the controller 60 may identify the start of the atrial refractory period 722 when it is desirable to charge the charge storage member during atrial activity. Optionally, the controller 60 may identify the start of the ventricular refractory period 822 when it is desirable to charge the charge storage member during ventricular activity. The start times for the atrial and ventricular refractory periods 722 and 822 may be calculated from the P-wave and the R-wave, respectively.

Next, at 912, the controller 60 sets the start and end times for the initial charge period to align with the corresponding refractory period (722 or 822). At 914, the controller 60 initiates a charging operation. By timing the charging period with the refractory period, the IMD 10 limits any adverse effects that may result when current leaks from an IMD. The charge storage member 421 is charged during a cardiac refractory period in which at least the majority of the excitable cardiac tissues become immune to stimuli. Therefore, the controller 60 is preferably configured to synchronize the charging period of the charge storage member 421 with the atrial and/or ventricular events.

The controller 60 is configured to synchronize the charging periods of the charge storage member 421 within a ventricular charge window 836. The ventricular charge window 836 extends for a predetermined or programmable period, i.e., approximately 0 msec to 40 msec following the detection of the R-wave, but still within the absolute ventricular refractory period 822. Advantageously, the excitable cardiac tissues do not respond to the stimulus during the ventricular charge window 836 because the ventricles are refractory and thus are not responsive to stimuli such as a stimulus resulting from a current leakage.

The same applies to the atrial however because the ventricular chamber takes priority in synchronization it may be preferred to first synchronize to an R-wave and then make charge out of the atrial vulnerable period.

Numerous variations of the present systems and methods exist without deviating from the scope of the present invention. For example, multiple leakage detection systems may be provided at different locations of the IMD 10 to detect leakage currents at these locations. For example, multiple switch banks may be implemented in parallel such that each switch bank may be disposed between the charge storage member and each implanted electrode. The controller may measure the voltage of the charge storage member, the rate of increase in the voltage, and/or the current flowing through respective current sensing members. When a leakage current exceeding the preset limit is detected, the controller terminates the charge supply to the leaking electrodes, while maintaining normal operation of the non-leaking electrodes.

FIG. 10 illustrates a post-leak assessment process 1000 performed by the controller 60 after leakage has been confirmed in accordance with an embodiment. The process 1000 begins when a leak is detected at 1002. Once leakage is detected, the leakage detection module 63 issues a visual and/or an audible warning signal at 1004 to the patient. The warning may also be physical such as a periodic vibration. At 1006, the leakage detection module 63 may determine whether the switch network 426 can be manipulated to minimize the impact of leakage. For example, the switch network 426 may be adjusted by opening or closing all, or one or more of the switches 452-455. Alternatively, if it is determined that only one switch is in-operative, the switch network 426 may no longer be able to deliver biphasic shocks. However, the switch network 426 may be able to deliver mono-phase shocks. Thus, the switch network 426 may be adjusted to a state where only mono-phase shocks can be delivered thereafter.

At 1008, the IMD 10 logs into memory any error information, the leakage measurements, IMD 10 status parameters, the state of various components within the IMD 10 at the time of leakage detection, recent cardiac activity, and the time at which leakage was detected. At 1010, the IMD 10 determines whether the state of the switch network 426 warrants one or more remedial actions. If the state of the switch network 426 does not warrant any remedial action, flow moves along 1012 and is done. However, if a remedial action is warranted, flow moves along 1014 and the IMD 10 disables the charging capability at 1016. At 1016 the charging and/or discharging process is terminated for the charge storage member 421, in order to eliminate the detected current leakage. When desirable, the leakage detection module 63 may also be configured to terminate a specific operation or the entire operation of the IMD 10 until a patient or an operator takes proper corrective actions. Accordingly, the leakage detection system 400 can limit the leakage of current to the patient even after the destruction of various electrodes 22, 23, 27, 28, 32, 34, 36, 38.

The leakage detection systems described herein generally seek to protect the patients from hazardous “strong” current spuriously leaking from the implanted electrodes. Other capacitors of the IMD, however, may also be utilized in detecting leakage of weak current which may gradually deplete the power cells of the IMD. Such capacitors may be selected to have high or low capacitance. Advantageously, upon selecting a desirable capacitor of the IMD, the foregoing leakage detection system and methods therefore can readily be implemented to the IMD 10 by reprogramming its controller to sense the voltage of the capacitor or to calculate the rate of change of its voltage. When desirable, an additional voltage sensor may be provided to sense the voltage of the capacitor. In an alternative embodiment, one or more capacitors may be added at desirable locations of the IMD, with the controller 60 sensing the voltage and/or rate of change of the added capacitors.

In yet another embodiment, the IMD 10 may be configured to minimize further leakage by suspending charging of the IMD, terminating the charging process of its charge storage member, and terminating operation of the IMD 10 until proper remedial action is taken. In particular, the controller may be configured to raise a warning flag, when the sensed voltage of the charge storage member after the pre-selected charging period does not reach the preset voltage or drops to a certain threshold after a wait period, when the sensed rate of increase in the voltage of the charge storage member is less than the preset rate, or when the leakage current sensed through the current sensing member exceeds the preset value. Upon detecting the warning flag, the IMD 10 which is provided with various warning systems configured to issue audio or vibratory warning signals to warn the patient or operator of current leakage, may issue various visual and/or audible warning signals to the patient and/or operator and/or log the error in its memory for future presentation to the following physician.

Optionally, the current sensor circuit may be at other locations within the circuitry of the IMD 10 as long as such the current sensor circuit senses the current flow between the charge storage member and the electrodes 28, 36, 38. In addition, because most current leakages tend to occur at, or near such electrodes, the current sensor circuit is generally placed in series with the leads 20, 24, 30.

In certain instances, the current leaking from a HV electrode may be less than 1 uA while the current leaking from a non-HV electrode being less than 0.1 uA. Accordingly, the controller 60 may be arranged to detect and to limit the current leakage that exceeds a predetermined limit. When current leakage is detected at such a low level as to pose no concern to the safety of the patient, in such instances the IMD 10 may not take an immediate corrective action. Instead, the low level current leakage may be permitted to continue without disabling charge. Even during low level current leakage, it may be desirable to warn the patient or notify a physician and to log certain information regarding the condition of the IMD.

Thus, various systems and methods therefore using leakage detecting and warning systems in implantable medical devices have been described in which currents and/or voltages are measured at various locations of such devices in order to detect leakage of current and patient warning signals are issued. While detailed descriptions of the specific embodiments of the present invention have been provided, it would be apparent to one of ordinary skill in the relevant art that numerous variations of the systems and methods described herein may be possible in which the concepts of the present invention may readily be applied and are not intended to be exclusive.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions, types of materials and coatings described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. §112, sixth paragraph, unless and until such claim limitations expressly use the phrase “means for” followed by a statement of function void of further structure.

What is claimed is:

1. A medical device, comprising:
a power source;
a charge storage member configured to receive and store energy from the power source;
a terminal connector configured to be coupled to a lead to be implanted in a patient proximate to tissue of interest,
a switch network electrically disposed between the charge storage member and the terminal connector, the switch network changing between open and closed states to disconnect and connect the charge storage member and the terminal connector;
a controller to control storage of energy in the charge storage member and delivery of stimulating pulses from the charge storage member to the lead coupled to the terminal connector; and
a leak detection module to obtain a leakage measurement by sensing at least one of i) a voltage potential of the charge storage member and ii) current flow from the charge storage member, the leak detection module compares the leakage measurement to a leakage threshold to determine when the leakage measurement satisfies the leakage threshold.

2. The device of claim 1, wherein the leakage detection module further comprises a current sensor disposed between the charge storage member and the switch network.

3. The device of claim 1, wherein the leakage detection module further comprises a current sensor disposed between the switch network and the terminal connector.

4. The device of claim 1, wherein the leakage detection module further comprises a voltage sensor disposed at the charge storage member to detect the voltage potential across the charge storage member.

5. The device of claim 1, wherein the leakage threshold constitutes a preset current range, the leak detection module identifying a current leak when the current flow is outside of the preset current range.

6. The device of claim 1, wherein the leakage threshold constitutes a preset voltage range, the leak detection module identifying a current leak when the voltage potential of the charge storage member is outside of the preset voltage range

7. The device of claim 1, wherein the charge storage member is configured to receive the energy during a refractory period of at least a portion of the tissues of a heart.

8. The device of claim 1, wherein the controller controls the charge storage member to receive the energy during a charging period which is synchronized with at least one of an atrial event and a ventricular event of a heart.

7. The device of claim 1, wherein the controller is configured to synchronize a charging period of the charge storage member with an R wave of the heart.

8. The device of claim 1, wherein the controller is configured to sense at least one of the voltage potential and current continuously.

9. The device of claim 1, wherein the controller is configured to sense at least one of the voltage potential and current intermittently.

10. The device of claim 1, wherein the controller is configured to sense at least one of the voltage potential and current for a preset charging period.

11. The device of claim 1, further comprising a current sensing member that includes a diode disposed in parallel with a resistor to sense the current flow to or from the switch network.

12. The device of claim 1, further comprising a current sensing member that includes at least two diodes disposed in parallel with a resistor, the diodes being disposed to allow current flow in opposite directions, the resistor to sense the current flow to or from the switch network.

13. The device of claim 1, wherein the controller decouples the charge storage member from the power source upon detecting that the leakage measurement exceeds the leakage threshold.

14. The device of claim 1 further comprising an electrode operationally coupled to the charge storage member and the tissue, the electrode configured to deliver the energy from the charge storage member to the tissues, wherein the controller is configured to operationally decouple the electrode from the charge storage member upon detecting that the leakage measurement exceeds the leakage threshold.

15. The device of claim 1, wherein the controller is configured to issue at least one of a vibratory warning signal and an audible warning signal upon detecting the leakage measurement exceeds the leakage threshold.

16. A method of detecting energy leakage from a medical device, comprising:
initiating charge of a charge storage member in the medical device;
sensing at least one of i) a voltage potential of the charge storage member and ii) current flow from the charge storage member, to obtain a leakage measurement;
comparing the leakage measurement to a leakage threshold to determine when the leakage measurement exceeds the leakage threshold; and
identifying energy leakage when the leakage measurement satisfies the leakage threshold.

17. The method of claim 16, further comprising terminating charging of the charge storage member upon identifying energy leakage, and operationally decoupling the charge storage member from the tissue upon identifying energy leakage.

18. The method of claim 16, further comprising operationally decoupling the electrode from the charge storage member upon identifying energy leakage.

19. The method of claim 16, further comprising determining a refractory period of at least a portion of the tissue of interest and timing the initiating operation to begin charging the charge storage member during the refractory period.

20. The method of claim 16, further comprising charging the charge storage member for a charging period ranging from about 20 msec to about 50 msec. before the sensing operation.

21. The method of claim 16, further comprising synchronizing the initiating operation with at least one of an atrial event and a ventricular event of a heart.

22. The method of claim 16, further comprising synchronizing the initiating operation with an R wave of the heart.

23. The method of claim 16, further comprising measuring at least one of the voltage potential and the current for a preset period continuously.

24. The method of claim 16, further comprising measuring at least one of the voltage potential and the current for a preset period intermittently.

25. The method of claim 16, wherein the sensing operation comprises monitoring the current flowing in both directions between the tissue of interest and the charge storage member.

26. The method of claim 16, wherein the comparing operation comprises:
obtaining at least one of an average voltage potential and average current flow over a preset period; and
comparing the at least one of the averaged voltage potential and average current flow to a preset voltage range and preset current range, respectively.
